(19) United States
(12) Patent Application Publication
(10) Pub. No.: US 2023/0215552 A1
(43) Pub. Date: Jul. 6, 2023

(54) EARLY DETECTION OF PATIENTS FOR COORDINATED APPLICATION OF HEALTHCARE RESOURCES BASED ON BUNDLED PAYMENT

(71) Applicant: CERNER INNOVATION, INC., NORTH KANSAS CITY, MO (US)

(72) Inventors: Vadim KHOTILOVICH, Leawood, KS (US); Andrew ROBERTS, Overland Park, KS (US); Will ZIMMERMAN, Kansas City, KS (US); Andrew WINDLE, NORTH KANSAS CITY, MO (US)

(21) Appl. No.: 18/146,931

(22) Filed: Dec. 27, 2022

Related U.S. Application Data
(60) Provisional application No. 63/295,727, filed on Dec. 31, 2021.

(51) Int. Cl. G16H 40/20; G16H 10/60

(52) U.S. Cl. CPC G16H 40/20; G16H 10/60

(57) ABSTRACT

Systems, methods, and computer-readable media are provided for predicting patient qualification for application of coordinated healthcare resources. In aspects, an indication of a patient encounter associated with a target patient is received. Feature values may be extracted from an electronic health record of the target patient stored in an electronic health record system. The extracted feature values may be input into a trained machine learning model to programatically determine whether the target patient qualifies for application of coordinated healthcare resources. Based on a determination that the patient qualifies for application of coordinated healthcare resources, a care protocol, which may include a coordinated allocated of resources, associated with application of coordinated healthcare resources may be initiated.

### CROSS-REFERENCE TO RELATED APPLICATIONS

This application claims the benefit of U.S. Provisional Pat. Application No. 63/295,727, filed on Dec. 31, 2021, titled “EARLY DETECTION OF PATIENTS FOR COORDINATED APPLICATION OF HEALTHCARE RESOURCES BASED ON BUNDLED PAYMENT.” The aforementioned application is assigned or under obligation of assignment to the same entity as this application, and is incorporated in its entirety in this application by reference.

### BACKGROUND

Allocation of resources for patient care is often dependent on many factors. One such factor is the patient’s eligibility for application of coordinated healthcare resources and bundled payments. Traditional reimbursement plans treat heath care providers separately based on individual services provided during a course of treatment associated with each of the healthcare providers. These traditional plans have resulted in fragmented patient care due to the lack of coordination across the healthcare providers associated with various healthcare settings. For example, these traditional plans focus on a quantity associated with patient services rather than the quality that patients are receiving.

Additionally, Bundled Payments for Care Improvement Advanced (BPCI Advanced) has been implemented as a new iteration of the Centers for Medicare & Medicaid Services (CMS) and the Center for Medicare and Medicaid Innovation (Innovation Center) for continuing efforts to enhance support to patients to better coordinate and improve the quality of healthcare. For example, participating healthcare providers receive a portion of a single payment bundled for multiple services and/or providers. Further, care providers may institute certain treatment protocols for a patient who is eligible for application of coordinated healthcare resources or a bundled payment to provide more coordinated care. However, identification of patient eligibility is often made months after a patient is discharged or months after a completion of an outpatient procedure. This delay in bundle identification and determination results in delayed patient-access to coordinated healthcare and reduced patient healthcare quality. Additionally, these delays may cause a worsened healthcare condition for a patient, which could have been prevented if the patient had received coordinated care after being identified as eligible for application of coordinated healthcare resources or a bundled payment.

### SUMMARY

Systems, methods, and computer-readable media are provided for implementing a decision support tool for early identification of patients who are eligible for coordinated resource allocation due to application of coordinated healthcare resources or bundled payment eligibility. In aspects, a patient encounter associated with a target patient may be detected. In some aspects, the patient encounters are identified as potentially qualifying for the coordinated resource allocation for bundle payment eligibility. In some aspects, a patient encounter is associated with one or more qualifying episodes for the application of coordinated healthcare resources or bundled payment eligibility. A patient encounter may include, for example, a video session between a patient and a medical practitioner or a medical staff member. A patient encounter may also include an inpatient or outpatient admission, or another type of in-person contact between a patient and a medical practitioner or a medical staff member. A patient encounter may also include performance or an interpretation of a test (e.g., a diagnostic test). In some embodiments, a patient encounter includes a full course of an inpatient stay at a healthcare facility or a stay at an emergency department.

Further, feature values, such as values for administrative data (e.g., information about past and current encounters), demographics, conditions (i.e., diagnoses), medications, lab tests results, and vitals features, may be extracted from patient data of the target patient. A trained machine learning model may be used to determine whether the target patient qualifies for application of coordinated healthcare resources (e.g., comprising a bundled payment) and, thus, coordinated care protocol(s) based on the extracted feature values. The machine learning model may determine eligibility scores for a plurality of clinical episodes associated with application of coordinated healthcare resources or bundled payment, where each eligibility score indicates a likelihood that the encounter of the target patient is for a given clinical episode. In some aspects, a ranking of the clinical episodes is made for the target patient based on the eligibility scores. Based on a determination that the target patient qualifies for application of coordinated healthcare resources or bundled payment based on at least one clinical episode, one or more actions may be initiated, such as notification of patient eligibility to a caregiver and, in some implementations, instituting a particular care protocol. In aspects, the machine learning model is trained using a plurality of feature values extracted from historical patient data and labels indicating the clinical episode for patient encounters in the historical patient data.

### BRIEF DESCRIPTION OF THE DRAWINGS

The present invention is described in detail below with reference to the attached drawing figures, wherein:

FIG. 1 depicts aspects of an illustrative operating environment suitable for practicing an embodiment of the disclosure;

FIG. 2 depicts an example timeline in which a patient eligibility predictor may be implemented, in accordance with an embodiment of the disclosure;

FIG. 3 depicts an example table listing the types of clinical episodes, in accordance with an embodiment of the disclosure;

FIG. 4 depicts an example table of metrics considered for feature selection, in accordance with an embodiment of the disclosure;

FIG. 5 depicts an example graph illustrating feature importance for an aggregated set of features used in a patient eligibility predictor, in accordance with an embodiment of the disclosure;

FIGS. 6A-6P depict example graphs illustrating a top subset of features, based on total gain, for each class for which a patient eligibility predictor makes an eligibility prediction, in accordance with an embodiment of the disclosure;

FIGS. 7A and 7B depict example graphs illustrating aspects of predictive performance of an example patient eligibility predictor, in accordance with an embodiment of the disclosure;

FIG. 8 depicts a flow diagram of an example method for a patient eligibility predictor, in accordance with an embodiment of the disclosure; and

FIG. 9 depicts an example embodiment of a computing device, in accordance with embodiments of the present disclosure.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

In addition, words such as “a” and “an,” unless otherwise indicated to the contrary, may also include the plural as well as the singular. Thus, for example, the constraint of “a feature” is satisfied where one or more features are present. Furthermore, the term “or” includes the conjunctive, the disjunctive, and both (a or b thus includes either a or b, as well as a and b).

As one skilled in the art will appreciate, embodiments of the invention may be embodied as, among other things: a method, system, or set of instructions embodied on one or more computer readable media. Accordingly, the embodiments may take the form of a hardware embodiment, a software embodiment, or an embodiment combining software and hardware. In one embodiment, the invention takes the form of a computer-program product that includes computer-usable instructions embodied on one or more computer readable media.

Accordingly, at a high level, this disclosure describes, among other things, methods and systems for automatic and early identification of whether a patient qualifies for bundled payment or any associated coordination of care resource allocation. In aspects, a patient eligibility predictor determines whether the patient is eligible for application of coordinated healthcare resources or bundled payment prior to the patient being discharged (from a hospital for example). Early predictions as to whether the patient qualifies for the application of coordinated healthcare resources or bundled payment enables improved quality of care.

In some embodiments, the methods and systems using the patient eligibility predictor may be implemented as a computer application or tool for indicating that a patient qualifies for the bundled payment and corresponding coordination of care resources. For example, a patient may enter a healthcare facility, thereby initiating a patient encounter with healthcare providers. In other implementations, the patient may begin a video session with the healthcare provider, initiating the patient encounter as well. The patient eligibility predictor detects the patient encounter and extracts feature values from the patient’s data in an electronic health record (EHR). In aspects, the EHR is stored in an EHR system that stores a plurality of EHRs for a population of patients. In aspects, the plurality of EHRs are initially received from a plurality of healthcare facilities.

Upon extracting the feature values from the patient’s data, a trained machine learning model is used to determine whether the patient qualifies for application of coordinated healthcare resources or bundled payment based on the feature values extracted from the patient’s EHR data. In aspects, the machine learning model comprises a multiclass classification model. The model may identify a likelihood of the patient having one or more types of clinical episodes (such as seizures, sepsis, congestive heart failure, stroke, and other clinical episodes for example, more of which are referenced herein) that are associated with a application of coordinated healthcare resources or bundled payment. Furthermore, the patient eligibility predictor may provide indications for display that indicate whether the patient qualifies for the application of coordinated healthcare resources or bundled payment, either overall or for a particular clinical episode. In some aspects, the patient eligibility predictor displays a ranked list of types of clinical episodes associated with application of coordinated healthcare resources or bundled payments for which the patient may be eligible. In aspects, upon receipt of the bundled payment, the bundled payment is divided across a plurality of providers associated with the bundled payment. In aspects, upon application of the coordinated healthcare resources, the bundled payment is divided across a plurality of providers associated with the bundled payment.

Currently available methods and systems comprise various problems, including delayed patient treatment and care due to inaccurate payment determinations and predictions, much slower and delayed payment determinations and predictions, and wasteful consumptions of processing resources for making determinations based on high volumes of data having low importance values associated with the determinations and predictions. For example, the delayed determinations of whether a patient is eligible for bundled payment are sometimes provided months after a patient is discharged or months after a completion of an outpatient procedure. At that time, it may be too late to institute certain treatment protocols associated with bundled payments that are intended to improve care coordination and, thus, improve patient outcome. Further, making determinations of patient eligibility that are either unreliable or delayed (i.e., too late in the care process to effectively provide coordinated care resource allocation) inefficiently consume computing resources, such as processing power, network bandwidth, throughput, memory consumption, etc. In some instances, predictions provided by the currently available methods and systems may even completely fail to satisfy a user’s (e.g., a healthcare provider’s) intended goal to reliably classify the patient, thus requiring even more time and computing resources.

To combat these problems with the currently available methods and systems, patients who qualify for one or more bundled payments must be promptly and reliably identified early in the encounter and administered corresponding coordinated care to reduce the possibility of further complications. Accordingly, embodiments of the present disclosure aim to reliably, promptly, and automatically identify a patient who qualifies for application of coordinated healthcare resources or bundled payment based on a particular clinical episode. Specifically, embodiments include receiving an indication of a patient encounter associated with a target patient. Further, patient data of the target patient is extracted from an EHR. The extracted patient data is then input into a machine learning model, such as a multiclass classification model, for example. Based on an output from the machine learning model, a determination is made as to whether the target patient qualifies for application of coordinated healthcare resources or a bundled payment.

One goal for the embodiments of this disclosure is to improve upon the problems with the current available technologies discussed above. For example, embodiments of this disclosure improve the quality and effectiveness of patient care by more reliably determining whether a patient qualifies for application of coordinated healthcare resources or a bundled payment early during an encounter. Further, embodiments of this disclosure improve upon the usage of processing resources for making such determinations. For example, rather than providing determinations months after a patient is discharged or months after a completion of an outpatient procedure, embodiments of the present disclosure provide reliable determinations automatically and prior to discharging the patient. In some embodiments, the determination of patient eligibility may be made before coding information for determining a Diagnosis Related Group (DRG) for the target patient’s encounter becomes available. In response to the early identification of eligible patients, embodiments of the present disclosure may subsequently provide for initiating a particular care protocol associated with application of coordinated healthcare resources or the bundled payment, which accordingly results in prompt appropriate and coordinated care for the patient and thereby improved patient outcomes.

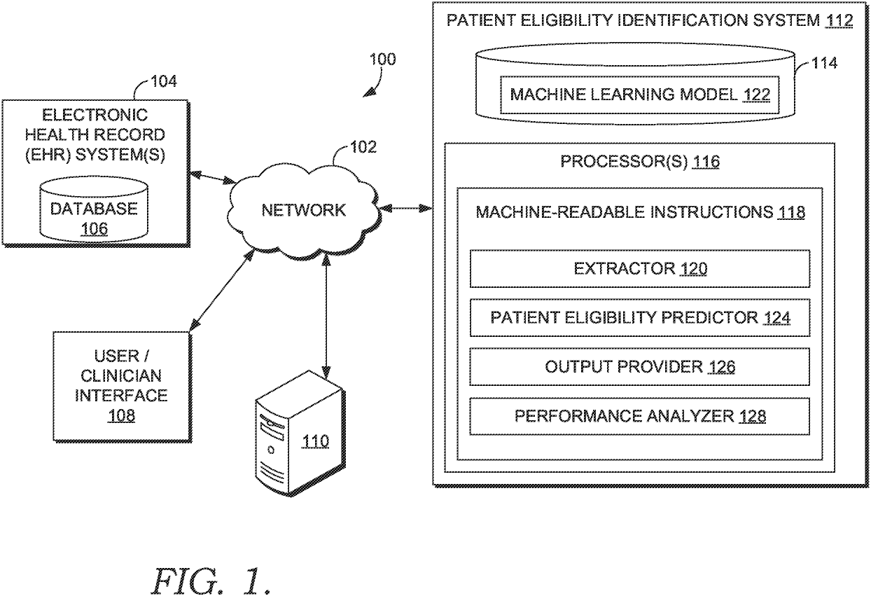

Referring now to the drawings generally and, more specifically, referring to FIG. 1, an aspect of an example operating environment 100 is provided suitable for practicing an embodiment of this disclosure. Certain items in block-diagram form are shown more for being able to reference something consistent with the nature of a patent than to imply that a certain component is or is not part of a certain device. Similarly, although some items are depicted in the singular form, plural items are contemplated as well (e.g., what is shown as one database might really be multiple databases distributed across multiple locations). However, showing every variation of each item might obscure aspects of the present disclosure. Thus, for readability, items are shown and referenced in the singular (while fully contemplating, where applicable, the plural).

As shown in FIG. 1, example operating environment 100 provides an aspect of a computerized system for compiling or running an embodiment of a patient eligibility predictor for determining whether a patient qualifies for a bundled payment and, in some aspects, providing corresponding coordination of care resources for the eligible patient. Example operating environment 100 includes a network 102 communicatively coupled to one or more EHR systems 104 comprising a database 106, a user/clinician interface 108, computer system 110, and patient eligibility identification system 112. As depicted by example operating environment 100, patient eligibility identification system 112 comprises a database 114 (which includes a machine learning model 122), processor(s) 116, and machine-readable instructions 118 that perform operations when executed on a computerized decision support system. For example, the operations may comprise actions performed by an extractor 120, a patient eligibility predictor 124, and an output provider 126. Some embodiments may further comprise a performance analyzer 128.

In some embodiments, components of example environment 100 that are shown as distinct components (e.g., machine learning model 122) may be embodied as part of or within other components of environment 100. For example, machine learning model 122 may comprise a plurality of models used in conjunction with rules-based logic and may be utilized by patient eligibility predictor 124. As another example, the EHR system(s) 104 may comprise one or more EHR systems, such as hospital EHR systems, health information exchange EHR systems, ambulatory clinic EHR systems, or psychiatry/neurology EHR systems. Such EHR systems may be implemented in computer system 110, for example. Similarly, EHR system 104 may perform functions for two or more of the EHR systems (not shown).

Network 102 may comprise the Internet, or one or more public networks, private networks, or other communications networks (such as a cellular network or similar network) for facilitating communication among devices connected through the network 102. In some embodiments, network 102 may be determined based on factors such as the source and destination of the information communicated over network 102, the path between the source and destination, or the nature of the information. For example, intra-organization or internal communication may use a private network or virtual private network (VPN). Moreover, in some embodiments, items shown as being communicatively coupled to network 102 may be directly communicatively coupled to other items shown communicatively coupled to network 102.

In some embodiments, example operating environment 100 may include a firewall (not shown) between a first component and network 102. In such embodiments, the firewall may reside on a second component located between the first component and network 102, such as on a server (not shown), or reside on another component within network 102, or may reside on or as part of the first component.

Embodiments of EHR system 104 may include one or more databases 106 of EHRs of a plurality of patients, which may be stored on the one or more databases 106, and may further include one or more computers or servers that facilitate the storing and retrieval of EHRs comprising patient data. In some embodiments, EHR system(s) 104 may be implemented as a cloud-based platform or may be distributed across multiple physical locations. EHR system(s) 104 may further include record systems that store real-time or near real-time patient (or user) information, such as wearable, bedside, or in-home patient monitors, for example. Although FIG. 1 depicts an exemplary EHR system 104 that may be used for storing patient data, it is contemplated that an embodiment may also rely on supporting applications or monitors (not depicted) for storing and retrieving patient data from EHRs, such as patient data acquired from the monitors.

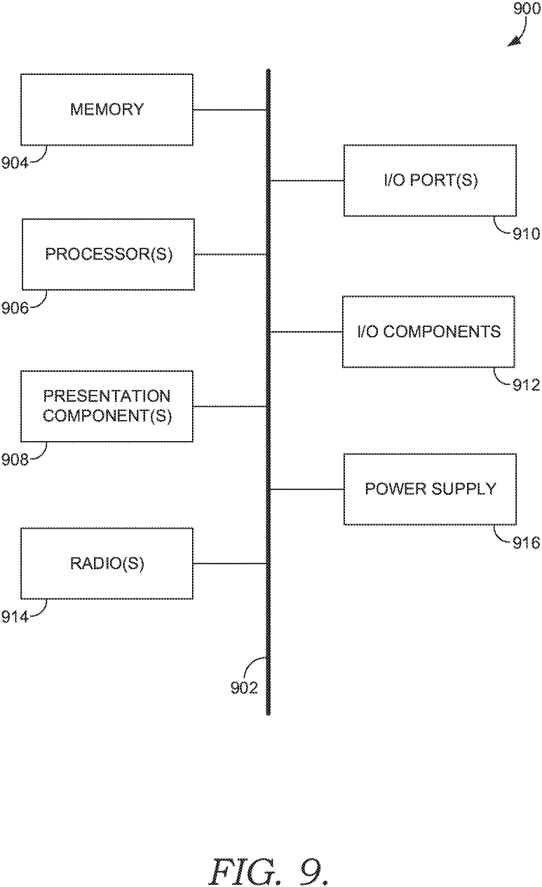

Example operating environment 100 further includes a user/clinician interface 108 communicatively coupled through network 102 to EHR system 104. Although environment 100 depicts an indirect communicative coupling between user/clinician interface 108 and EHR system 104 through network 102, it is contemplated that an embodiment of user/clinician interface 108 is communicatively coupled to EHR system 104 directly. An embodiment of user/clinician interface 108 takes the form of a graphical user interface operated by a software application or set of applications on a computing device (e.g., such as the computing system 900 depicted in FIG. 9).

In an embodiment, the software application or set of applications include PowerChart® software manufactured by Cerner Corporation. In an embodiment, the software application or set of applications comprise a Web-based application or applet. A healthcare provider application may facilitate accessing and receiving information from a user or healthcare provider about a specific patient or set of patients for which eligibility of application of coordinated healthcare resources or a bundled payment is identified, according to the embodiments presented herein. Embodiments of user/clinician interface 108 also facilitate accessing and receiving information from a user or healthcare provider about a specific patient (e.g., target patient) or population of patients including patient history; healthcare resource data; physiological variables (e.g., vital signs) measurements, time series, and patient eligibility predictions (including displaying the determined prediction(s) or initiating a treatment protocol associated with the bundled payment) described herein; or other health-related information, and facilitates the display of predictions, results, recommendations, treatments, or orders, for example. In an embodiment, user/clinician interface 108 also facilitates receiving orders, such as orders for more resources, from a user based on the eligibility predictions. User/clinician interface 108 may also be used for providing diagnostic services or evaluating the performance of the patient eligibility identification system 112.

An embodiment of the software application or set of applications (which may include programs, routines, functions, or computer-performed services) residing on a computing device, on one or more servers in the cloud, or distributed in the cloud and on a computing device (such as a personal computer, laptop, smartphone, tablet, mobile computing device, front-end terminals in communication with back-end computing systems or other computing device(s), such as computer system 110 described below). In an embodiment, the software application or set of applications include a Web-based application or applet (or set of applications) usable to provide or manage user services provided by an embodiment of the invention. For example, in an embodiment, the software application or set of applications facilitate processing, interpreting, accessing, storing, retrieving, and communicating information acquired from EHR system 104 or patient eligibility identification system 112, including predictions determined by embodiments of the patient eligibility identification system 112 as described herein. In an embodiment, the software application or set of applications transmit a recommendation or notification associated with the bundle eligibility prediction directly to user/clinician interface 108 through network 102. Further, some embodiments of the software application or set of applications utilize user/clinician interface 108 to facilitate access by a user (including a clinician/caregiver or patient) to functions or information on a graphical user interface, such as operational settings or parameters, user identification, user data stored locally on a computing device, and diagnostic services or firmware updates, for example.

In some embodiments, user/clinician interface 108 facilitates accessing and receiving information from a user or healthcare provider about a specific patient, a set of patients, or a population according to the embodiments presented herein. Such information may include historical data; health care resource data; variables measurements, time series, conditions, Diagnosis Related Groups (DRGs), lab tests, lab reports, lab results, vitals, medication, patient demographics, various patient encounter data, and bundle eligibility predictions; or other health-related information. User/clinician interface 108 also facilitates the display of results, recommendations, or orders, for example.

Example operating environment 100 further includes computer system 110, which may take the form of a server, which is communicatively coupled through network 102 to EHR system 104 and patient eligibility identification system 112. Computer system 110 may comprise one or more processors operable to receive instructions and process them accordingly. The computer system 110 may be embodied as a single computing device or multiple computing devices communicatively coupled to each other. For example, the multiple computing devices may comprise a server, desktop computer, laptop, tablet, cloud-computing device, a distributed computing architecture, ultra-mobile PC, or a mobile phone. In one embodiment, processing actions performed by computer system 110 are distributed among multiple locations, such as one or more local clients and one or more remote servers. In aspects, the processing actions may be distributed across the other components of example operating environment 100.

Example operating environment 100 further includes patient eligibility identification system 112, which is communicatively coupled through network 102 to EHR system 104, user/clinician interface 108, and computer system 110. Example patient eligibility identification system 112 comprises a database 114 (which may comprise a machine learning model 122), processor(s) 116, machine-readable instructions 118, an extractor 120, a patient eligibility predictor 124, and output provider 126. Some embodiments may further comprise a performance analyzer 128. It should be understood that, in some aspects, database 114, processor(s) 116, and/or machine-readable instructions 118 shown for patient eligibility identification system 112 are part of other components depicted in example operating environment 100, such as computer system 110. In aspects, the machine-readable instructions 118 may include media implemented in any method or technology for storing information (e.g., for storage in the database 114). Examples of storable information include computer-useable instructions, data structures, program modules, and other data representations. Machine-readable media may be associated with RAM, ROM, EEPROM, flash memory or other memory technology, CD-ROM, digital versatile discs (DVD), holographic media or other optical disc storage, magnetic cassettes, magnetic tape, magnetic disk storage, or other magnetic storage devices. For example, database 114 can store data momentarily, temporarily, or permanently.

Turning to extractor 120, extractor 120 may extract feature values from patient data, which may be in an EHR of the patient stored within the EHR system(s) 104. The feature values may be for features previously determined to be relevant to determining whether a patient’s encounter falls within a clinical episode (or group of clinical episodes) that is associated with a bundled payment. Determination of relevant features is discussed in greater details below with respect to machine learning model 122. Examples of features for which values may be extracted from patient data include features relating to demographics (e.g., age, gender, ethnicity, race, or insurance type), encounter history, conditions, medications, and physiological features such as lab tests and vitals. Feature values may be extracted from the information that was recorded within EHR up until the prediction time, including current and past encounters.

In some instances, the feature value may be extracted directly from the patient’s EHR. For example, quantitative lab result or vital (e.g., a first measurement of blood pressure during a current encounter) may be extracted from the EHR and used as a continuous feature value. In some instances, the feature value may be derived from information extracted from the patient’s EHR. For example, extractor 120 may assign a binary value for a particular category based on the patient’s data (e.g., assign a “1” if a patient has a diagnosis of acute myocardial infarction and assign a “0” if a patient does not for a diagnosis/condition feature value). Extractor 120 may also perform calculations using the patient’s extracted data to derive a feature value. For example, where a patient length of stay is not directly listed in the patient’s EHR, a length of stay value may be computed based on information indicating a date for the beginning of a stay and either the current date or the date of discharge, if discharge already occurred.

In one non-limiting example, the following features from the historical patient data were extracted for developing the machine learning model 122 of patient eligibility predictor 124 (as further described below):
Preferred Demographics
   3 features
   Types: boolean/binary
Encounters
   22 features
   Types: boolean /binary, continuous
Conditions
   933 features
   Time frames: current_2_days, past_2_to_30_days, past_2_to_365_days
   Types: boolean /binary, continuous
Medications + MedicationAdministration
   226 features
   Time frames: current_2_days
   Types: boolean /binary
Medications
   662 features
   Time frames: past_2_to_30_days, past_2_to_365_days
   Types: boolean /binary, continuous
Results + DocumentReferences
   4 features
   Time frames: current_2_days, past_30_days, past_365_days
   Types: boolean /binary
Results
   493 features
   Time frames: current_2_days, past_30_days, past_365_days
   Aggregations: max, median, min
   Types: boolean /binary, continuous
DRGs
   267 features
   Time frames: current_2_days, past_30_days, past_365_days
   Types: boolean /binary, continuous

Patient eligibility predictor 124 is generally responsible for determining, based on the feature values extracted from a patient’s data, a likelihood that the patient’s encounter is one of a select number of clinical episodes, or groups of clinical episodes, that correspond to application of coordinated healthcare resources or a bundled payment. In an embodiment, the one or more clinical episodes may each be associated with a standardized code, such as a Medicare Severity-Diagnosis Related Group (MS-DRG) code, an All Patient -Diagnosis Related Group (AP-DRG), a Healthcare Common Procedure Coding System (HCPCS) representing medical procedures, supplies, products and services, or another type of standardized code. In some aspects, the standardized code comprises a Global Surgery Code. Additionally, in some aspects, the standardized code may be associated with a clinical episode service provided by an Emergency Department, a clinical service provided by an episode initiator, or a clinical service provided by a non-episode initiator. In some embodiments, the standardized code defines a clinical episode type.

In aspects, the clinical episodes may be based on a diagnosis (e.g., chronic obstructive pulmonary disease) or a procedure (e.g., coronary artery bypass graft). Multiple diagnoses and/or procedures may be grouped together to represent a clinical episode. These diagnoses and/or procedures may each be represented by MS-DRG codes such that the clinical episodes may be groups of procedures and/or diagnoses having associated MS-DRG codes. In an example embodiment, clinical episodes represented by MS-DRG codes may include the following: acute myocardial infarction; back and neck (except spinal fusion); bariatric surgery; cardiac arrhythmia; cardiac defibrillator; cardiac valve; cellulitis; chronic obstructive pulmonary disease, bronchitis, and asthma; congestive heart failure; coronary artery bypass graft; disorders of the liver excluding malignancy; cirrhosis, or alcoholic hepatitis; double joint replacement of the lower extremity; fractures of the femur and hip or pelvis; gastrointestinal hemorrhage; gastrointestinal obstruction; hip and femur procedures excluding major joint; inflammatory bowel disease; lower extremity and humerus procedure excluding hip, foot, femur; major bowel procedure; major joint replacement of the lower extremity; major joint replacement of the upper extremity; pacemaker; percutaneous coronary infection; renal failure; seizures; sepsis; simple pneumonia and respiratory infections; spinal fusion; stroke; endovascular cardiac valve replacement; or urinary tract infection. Further, multiple MS-DRG codes may be associated with one of these clinical episodes. In aspects, the diagnosis or procedure forming the basis of the clinical episode occurs during an anchor encounter (such as an inpatient hospital stay).

Exemplary aspects of patient eligibility predictor 124 are generally configured to determine the likelihood that the patient’s encounter corresponds to application of coordinated healthcare resources or a bundled payment using a machine learning model, such as machine learning model 122. In some aspects, machine learning model 122 is a multiclass classification model. For example, machine learning model 122 may determine a likelihood that the patient has each of the predetermined types of clinical episodes that are associated with a bundled payment or coordinated care plan. For instance, patient eligibility predictor 124 may utilize machine learning model 122 to determine a likelihood that a patient is having an acute myocardial infraction episode, a likelihood that the patient is having a cardiac valve clinical episode, and a likelihood that the patient is having a cardiac arrhythmia clinical episode, among others. In some aspects, patient eligibility predictor 124 also determines a likelihood that the patient is not eligible for any of the predetermined clinical episodes associated with bundled payments or application of coordinated healthcare resources.

In other aspects, machine learning model 122 utilized by patient eligibility predictor 124 comprises one or more binary classification models. For example, in one aspect, machine learning model 122 includes a plurality of separate binary classification models, each of which correspond to a clinical episode. In aspects, machine learning model 122 uses a set of rules-based logic for determining whether a patient qualifies for a bundled payment and application of coordinated healthcare resources based on data extracted from EHR system(s) 104, such as condition data, MS-DRG data, or procedure data.

In aspects, the machine learning model 122 is trained based on feature values extracted from historical patient data of a reference population of patients. This data may be referred to herein as training patient data. In some aspects, the historical patient data is extracted from patient EHR records having MS-DRG codes (e.g., corresponding to patients who have insurance providers that provide for Bundled Payments for Care Improvement (BPCI) Advanced program (e.g., Medicare) and patients without such insurance providers). In some embodiments, the historical patient data includes similar types of data discussed with respect to extractor 120, such as demographics, encounter history, conditions, medications, and physiological features such as lab tests and vitals. This historical patient data may be from a current encounter (i.e., an encounter for which patient eligibility is being determined) and past encounters.

Training data selection criteria may be applied to identify the training population (which may also be referred to as a cohort) on which training will be performed. The selection criteria may include inclusion and/or exclusion criteria. In applying the training data selection criteria, in some aspects, the training data includes data from patients who had an ongoing inpatient hospital visit with a discharge date at least 90 days before the extraction of training data from EHR. As another example, training data selection criteria may exclude patients who have had an end stage renal disease eligible Medicare status. In some aspects, training data selection criteria may exclude patients whose encounter is 60 days or greater in length. In some aspects, training data selection criteria may include patients who are within a certain age range (e.g., patients over 18). In some aspects, training data selection criteria may exclude patients who had a positive COVID-19 diagnosis during the anchor encounter or within a 90-day post-discharge window.

In some aspects, the training data selection criteria excluded historical patient data for encounters that lacked information of a final diagnosis related group, which helped ensure that the historical patient data used for training was tied to a qualifying encounter associated with a diagnosis related group outcome. As one non-limiting example, the qualifying encounter may include a patient encounter within thirty days from training the machine learning model 122, and the qualifying encounter may be associated with a diagnosis related group outcome including a double joint replacement of the lower extremity. Further, in some aspects, training data selection criteria excludes historical patient data having a diagnosis related group that falls outside the BPCI Advanced program (e.g., historical patient data from a behavioral health facility). Note that while facility-based exclusions may apply in selecting training data, they may not apply in deployment of a trained model to allow for client flexibility of where eligibility scores may be produced.

The information of a final diagnosis related group may be used to label the training data so that each patient encounter represented in the training data would have a label of the most appropriate clinical episode, if any, that the encounter belongs to. This label acts as a ground truth for training the machine learning model 122. The labeled patient encounters may also be labeled based on DRGs, EHR data, or claims associated with a patient encounter. In some embodiments, the claims can be used to identify clinical episodes in historical EHR data. One non-limiting example may include EHR data involving a urinary tract infection clinical episode having MS-DRG codes 689 and 690.

Developing the machine learning model 122 also includes performing feature selection on the training data. Feature selection identifies a set of features that are the most relevant for reliably predicting patient eligibility for one or more clinical-episode based bundled payments utilizing the machine learning model(s). In aspects, one or more gradient boosted tree models may be used for determining features. In one embodiment, the one or more gradient boosted tree models may include a python package XGBoost model. In some aspects, the training data is initially split into binary and non-binary feature sets for feature selection in order to reduce bias against binary features due to one or more decision tree algorithms (e.g., a decision tree algorithm used by the XGBoost model).

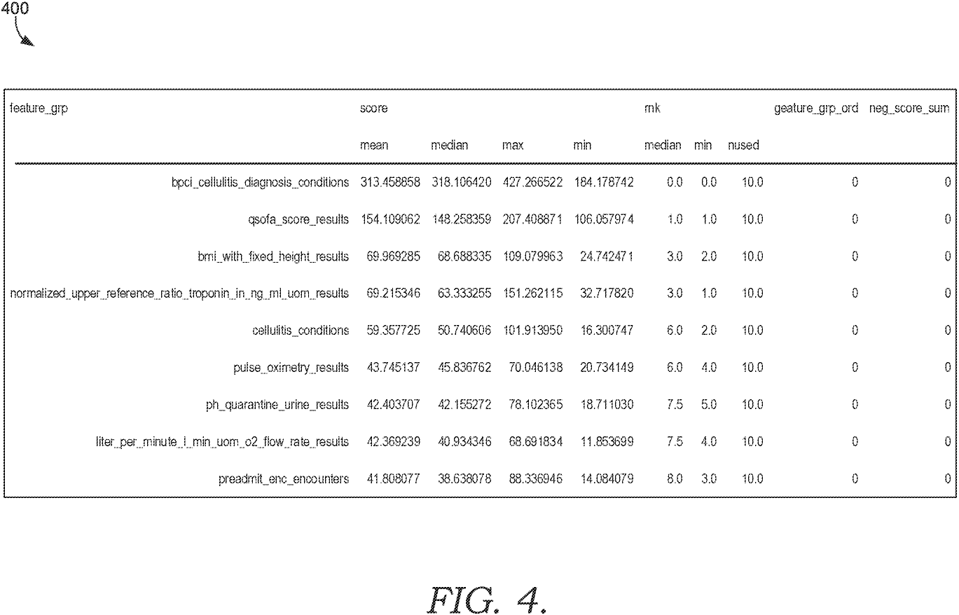

In one implementation, the data was further split into ten internal folds using stratified group splitting to keep prevalence of each of the different classes (i.e., types of clinical episodes) equal through the ten folds. Ten models may be trained on nine of the ten internal folds of training data, and the models were applied to the holdout fold (i.e., the tenth fold not initially used in the training) to provide an accurate estimate of the total gain that a feature can provide on unseen data. The total gain may then be aggregated across the ten models. Aggregation may occur by determining the mean total gain, the median total gain, the maximum total gain, or the minimum total gain in various implantations. In some aspects, the mean total gain was determined and used to order the features. In some aspects, the median rank of the total gain, the minimum rank of the total gain, and the number of models that a feature was used in was also determined and tracked for each feature. In example aspects, feature importance using these metrics was considered on a class-by-class basis, instead of looking at importance across all classes together, which would ensure that each class would be equally considered. FIG. 4 depicts table 400 that shows an example of information that may be tracked for each feature in feature selection. Table 400 represents only a subset of features.

Using the feature rankings/metrics, feature selection criteria may be applied. In some implementations, feature selection criteria includes a requirement that at least a predetermined minimum number (e.g., seven) of the cross-validation models used the feature, which ensures that a feature is important on a broad subset of data. Additionally or alternatively, no more than a predetermined maximum number of features (e.g., four) within a concept group may be considered important to any one class. For instance, no more than four features related to systolic blood pressure could be considered as important for one particular class, such as Stroke. This requirement encourages input diversity. Additionally or alternatively, only features with a count of features with a higher rank and a negative total gain on one or less holdout set are kept for feature selection. Further, only features with a mean total gain above the second feature with a negative total gain in some fold may be kept for feature selection.

In some aspects, there are datasets from multiple clients (e.g., multiple healthcare facilities) represented in the training data and the feature selection criteria, including the examples provided above, which may be applied for a class/client pair. In these instances, after the feature selection criteria is applied, features selected from each of the clients may be aggregated together.

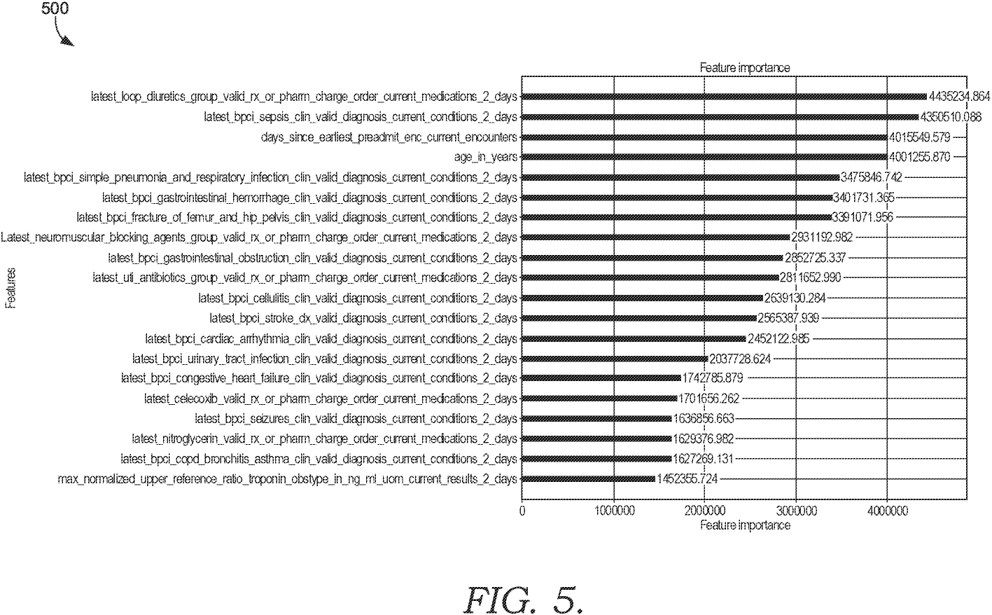

A top quantity of the ranked binary and continuous features for any class may be selected for continuation into a final model. In some aspects, this quantity is 20 such that the top 20 binary and continuous features for any class are selected for the final model. Further, in some aspects, any features that were considered important for a class at all different clients/data partitions may also be kept. Additionally, in some aspects, if any continuous features measuring data since a given concept appeared within the patient data is in the selected feature, any binary version of the same feature or shorter lookback periods that were selected may be removed. For example, if a feature looking at days since a malnutrition diagnosis within the last year is identified in the top features, a binary feature indicating a malnutrition diagnosis within the last year as well as malnutrition diagnosis within the last 30 days may be removed if those features were also included. FIG. 5 depicts a graph 500 illustrating top features using aggregated total gain across all classes (i.e., clinical episodes) that may be selected in accordance with the described feature selection process. It should be understood that this is merely an example of the top aggregated features in one implementation.

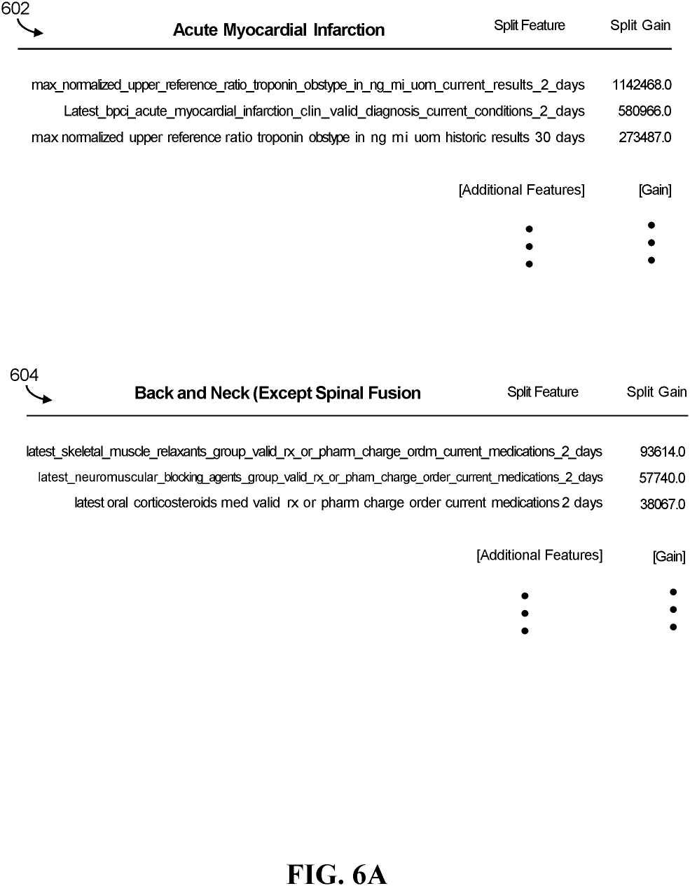

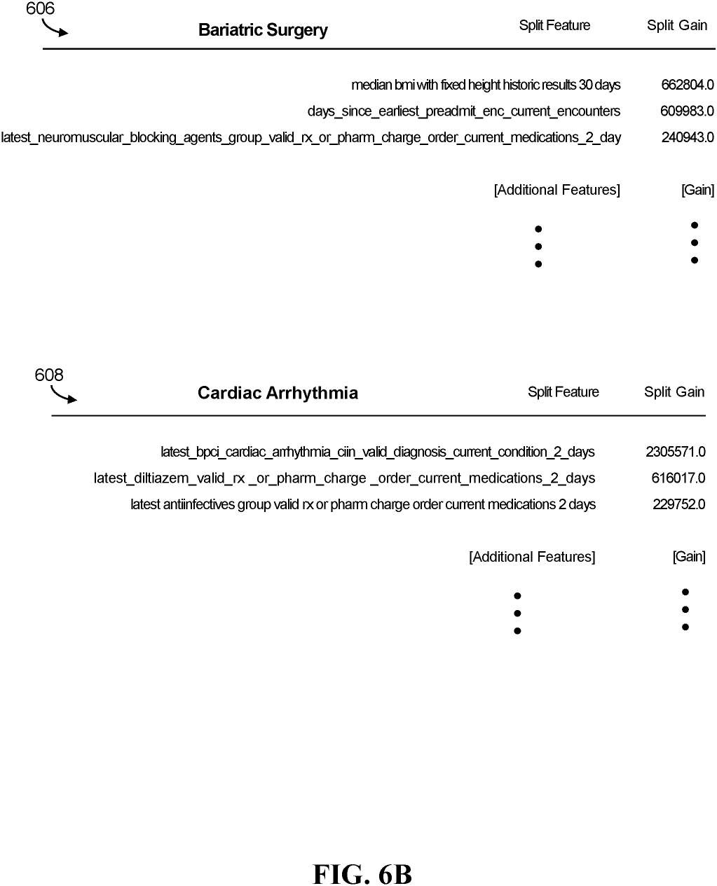

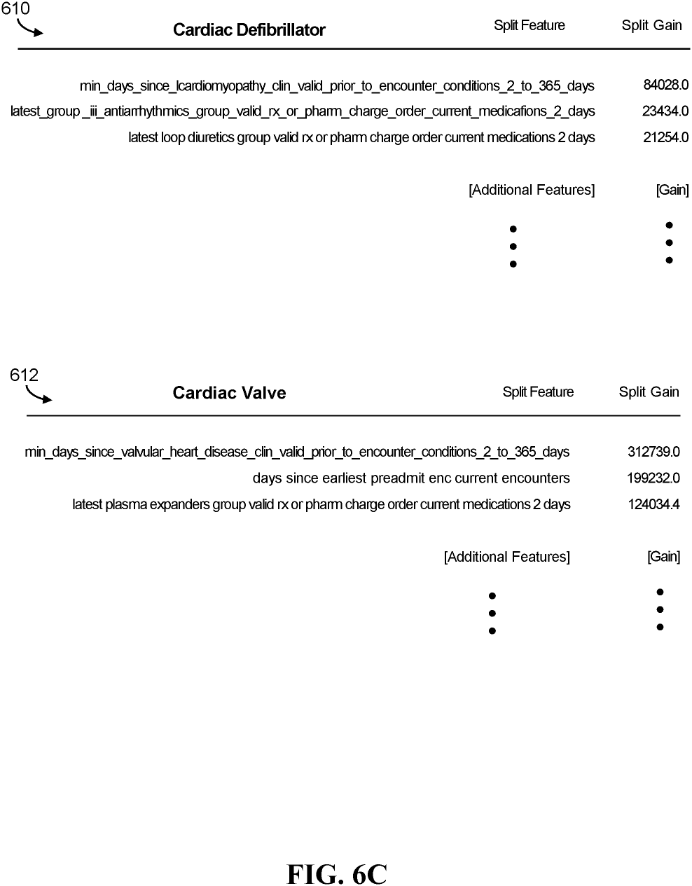

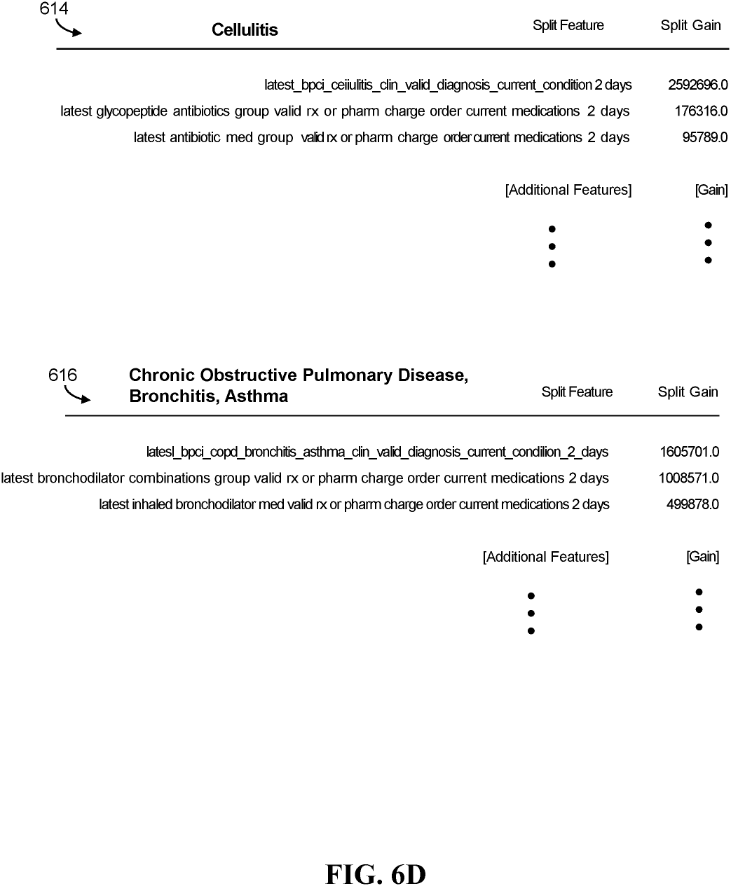

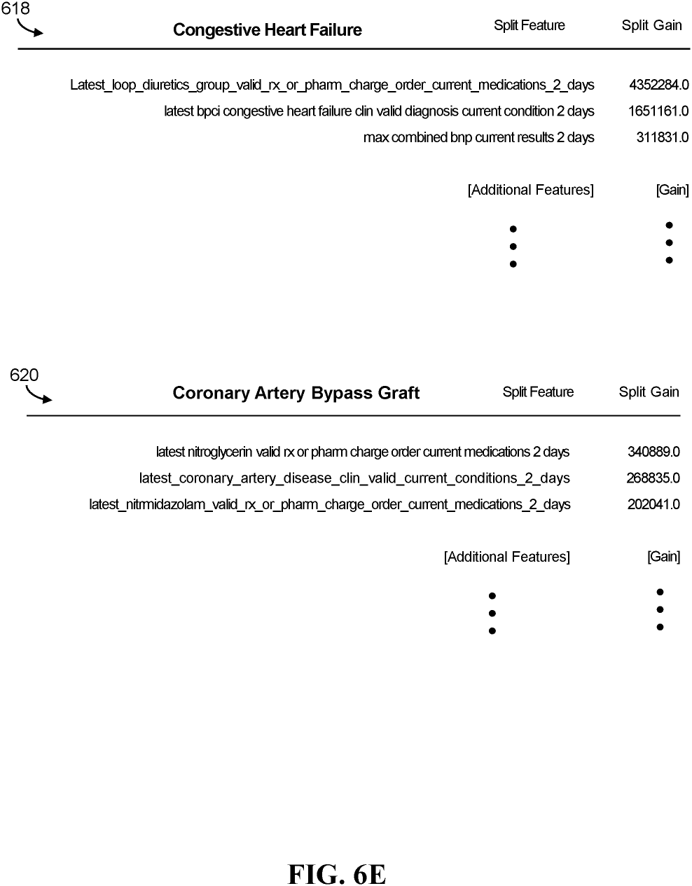

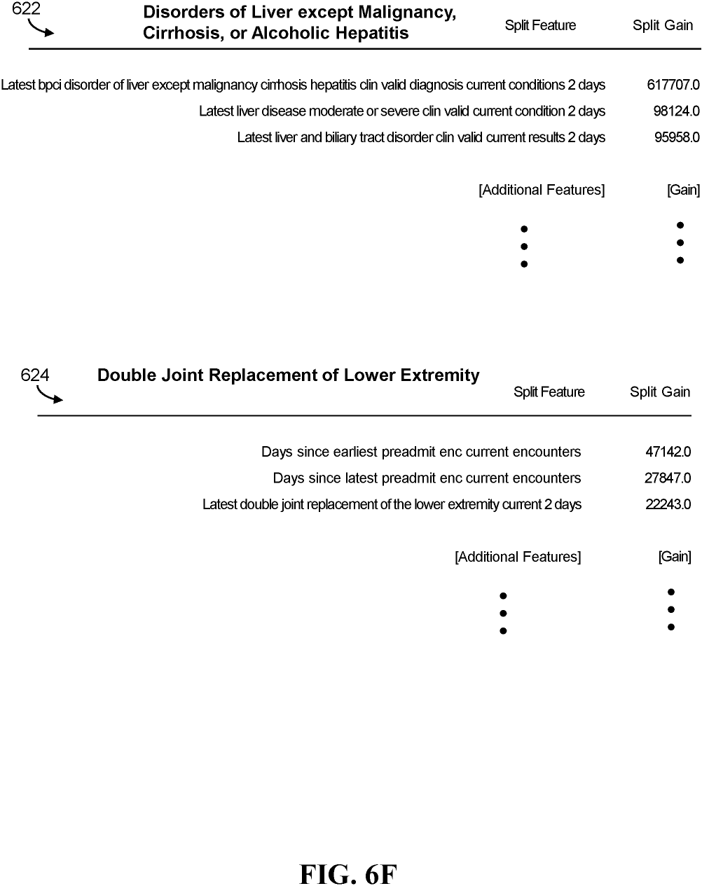

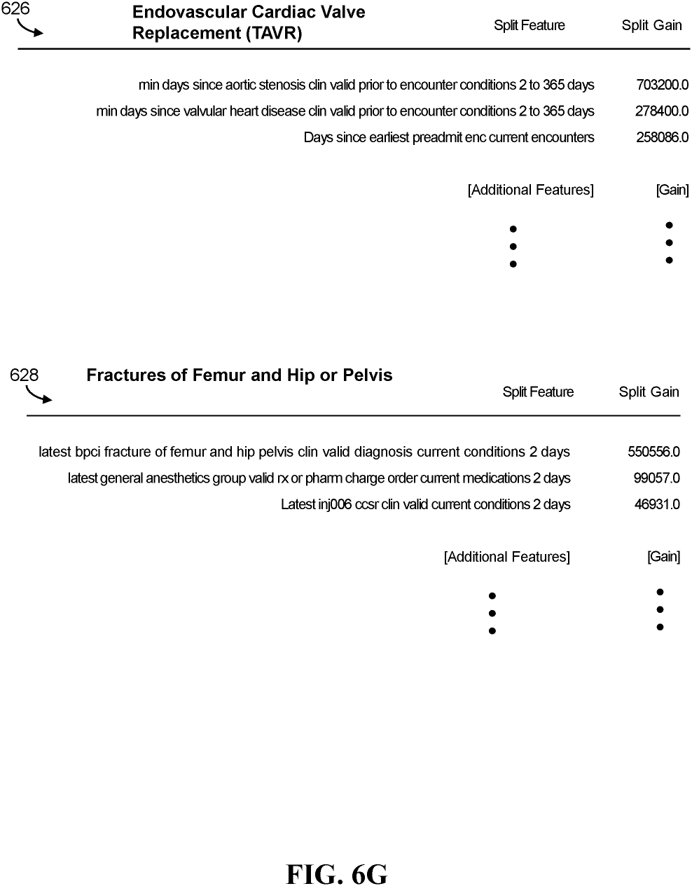

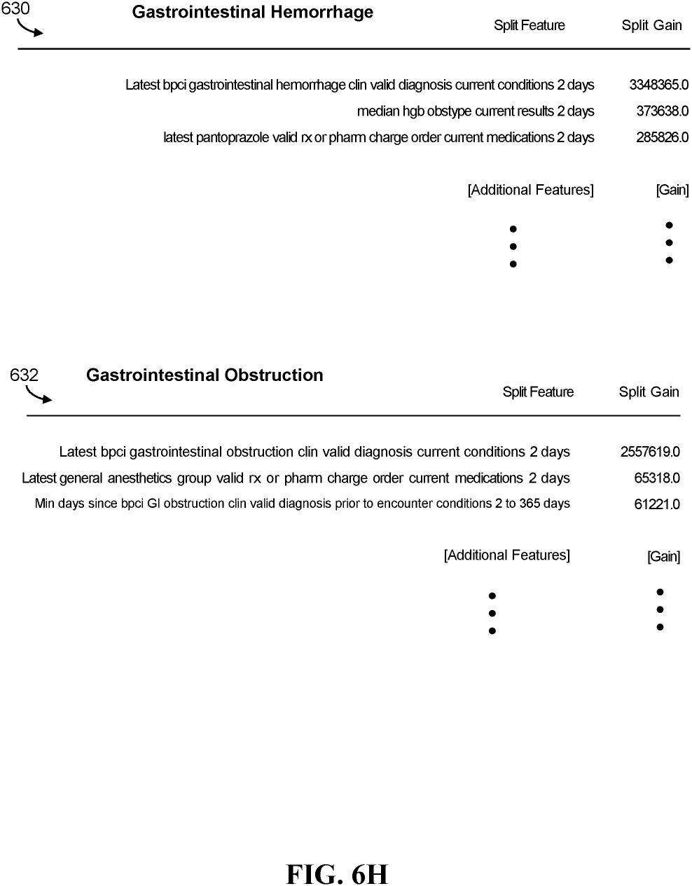

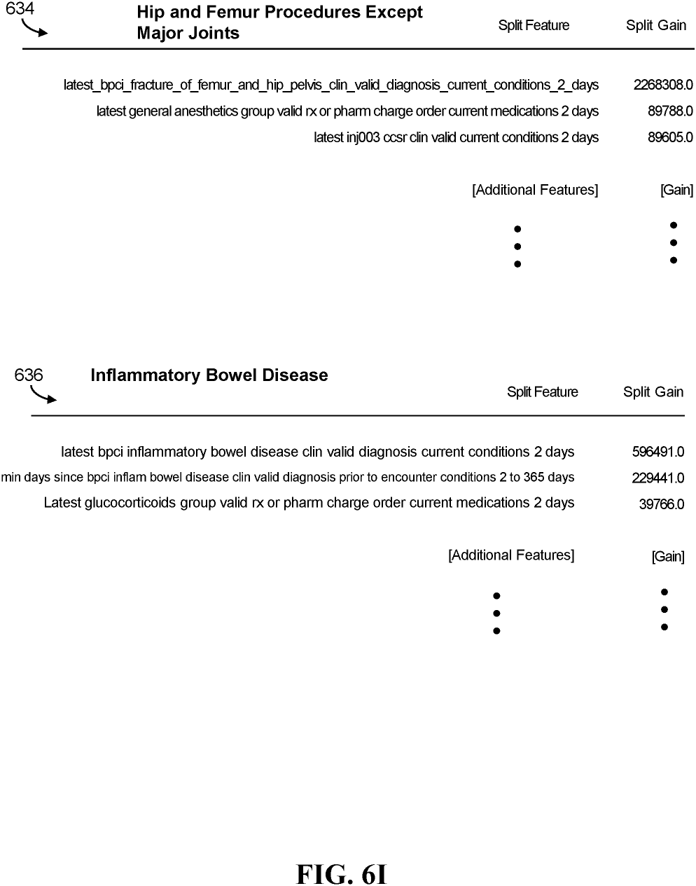

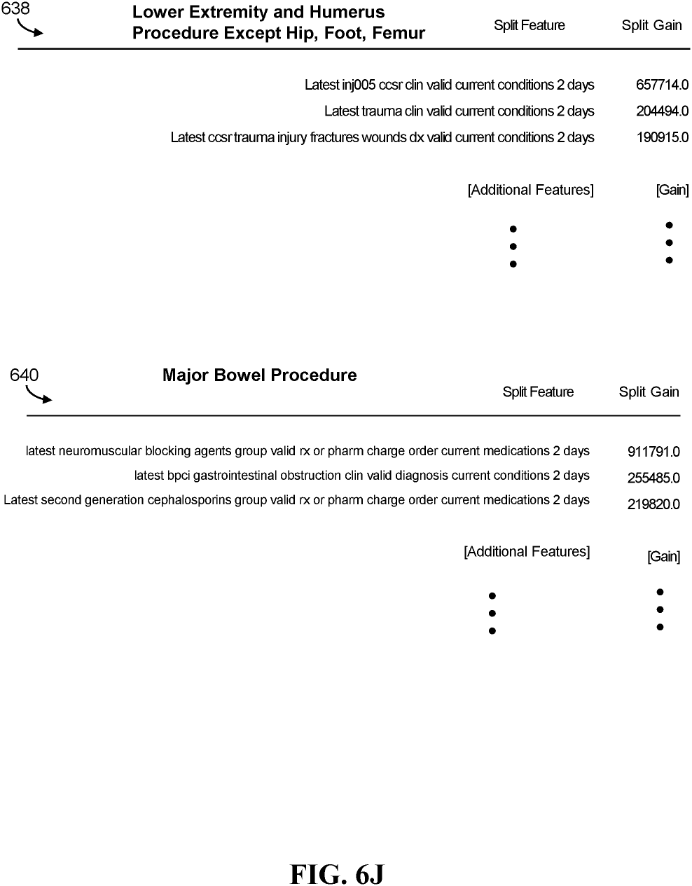

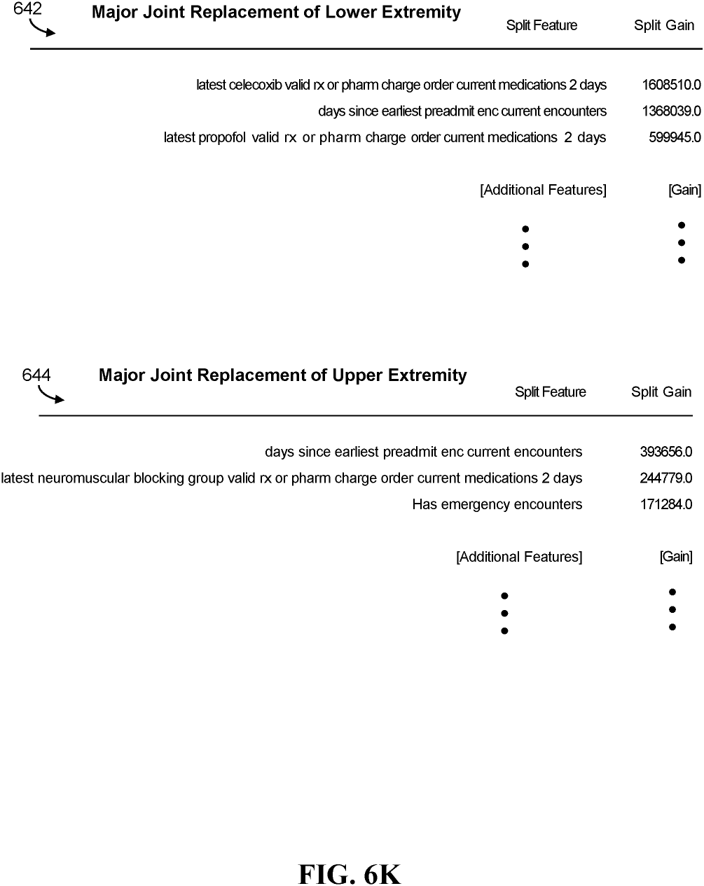

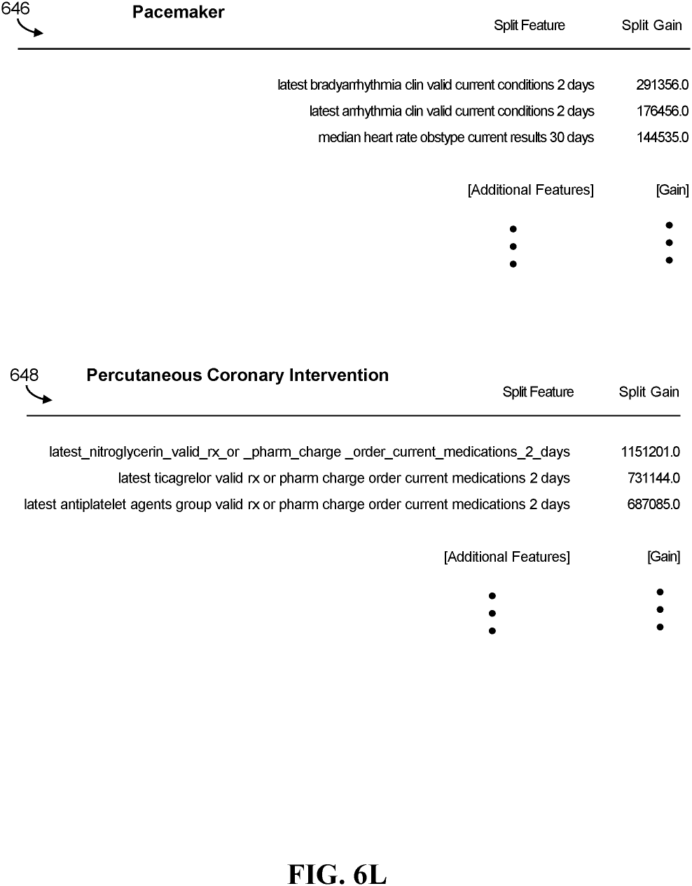

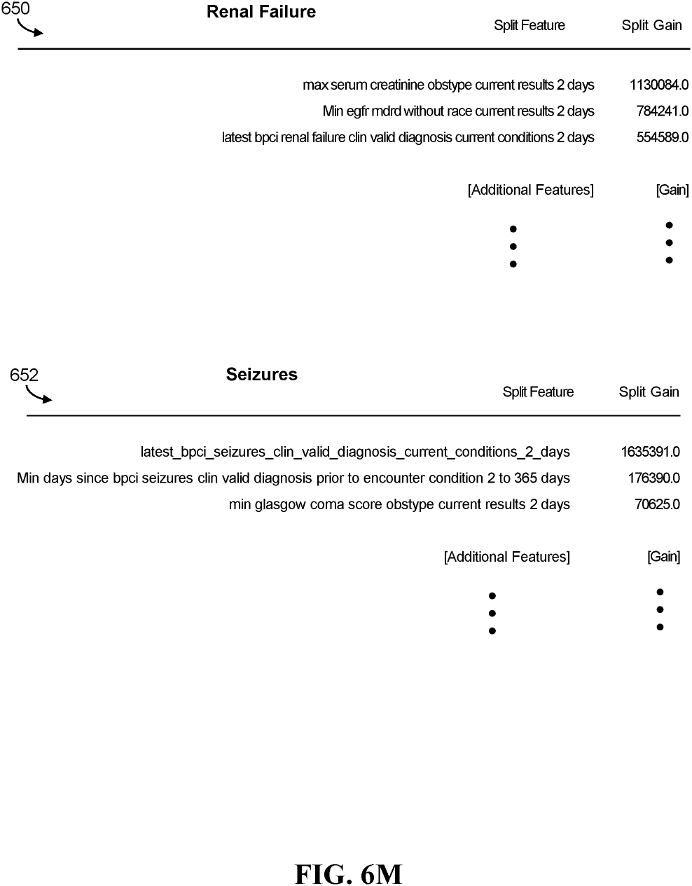

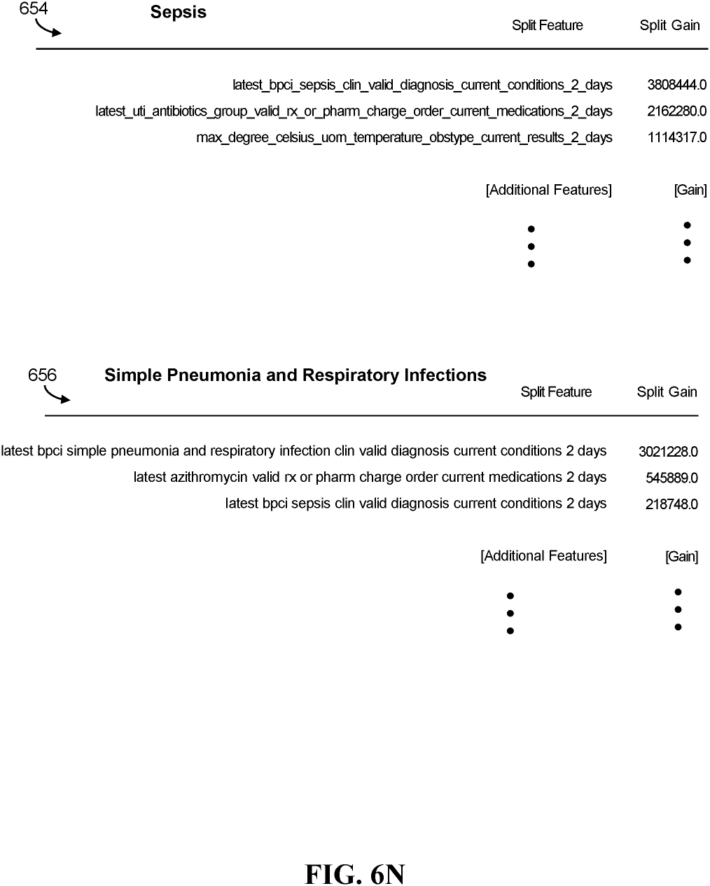

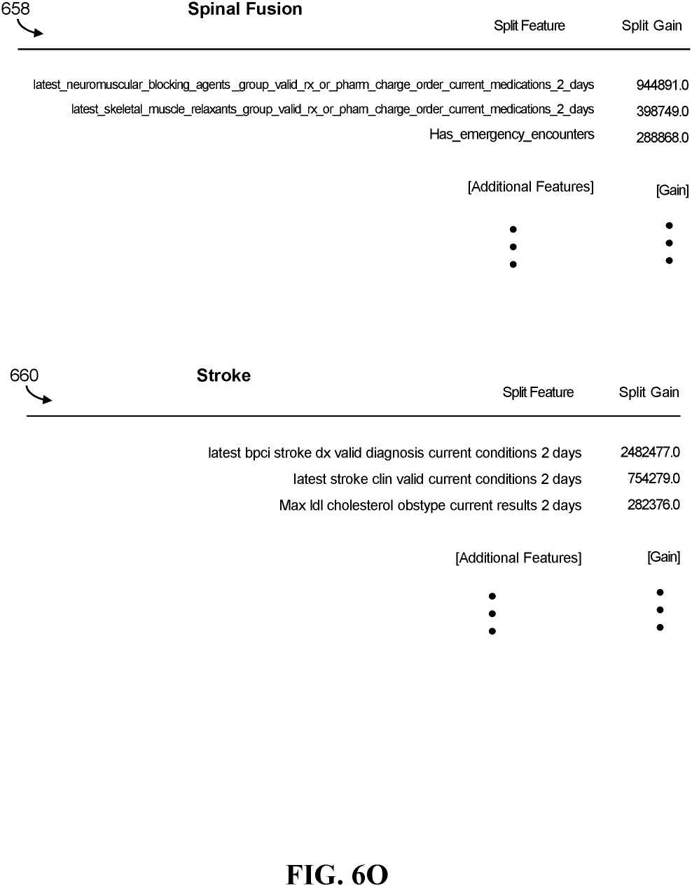

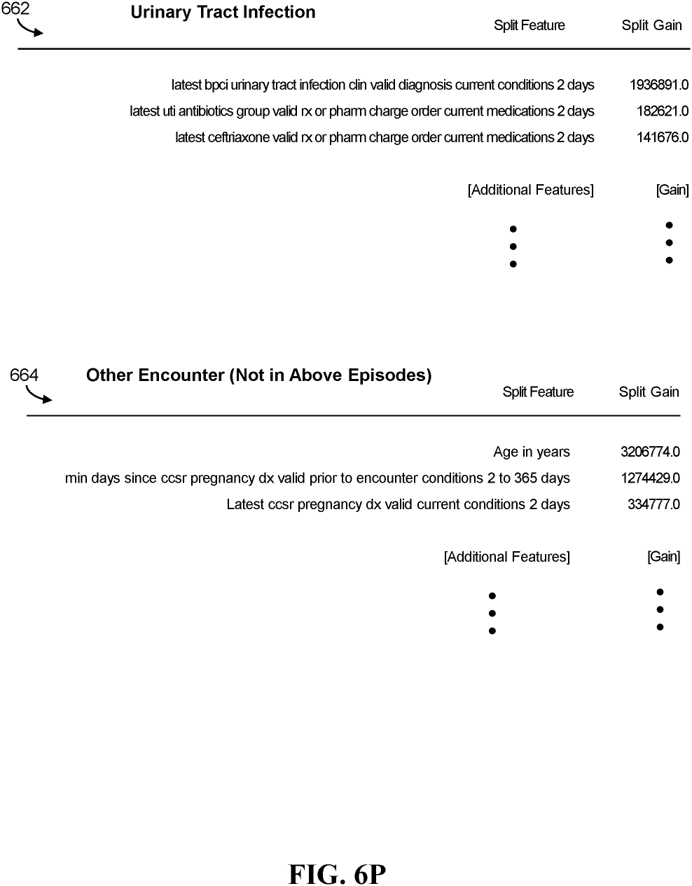

Further, there may be different top features utilized for each class (i.e., clinical episode) such that more features may be selected. In one example, approximately 600 features were selected for the final model to identify the likelihood of a patient being eligible for a bundled payment and application of coordinated healthcare resources for 31 particular clinical episodes and a likelihood of a patient not being eligible for any of the 31 clinical episodes. The top features based on total gain for each class in an example implementation are shown in tables 602-664 in FIGS. 6A-6P. Each of tables 602-662 are identified with a particular clinical episode, and table 664 is identified with no clinical episode. It should be understood that these tables in FIGS. 6A-6P are examples of top features in one implementation and that other features may be selected for other implementations. For example, Shapley Additive exPlanations (SHAP) values may be used as a metric for determining feature importance. In one aspect, total gain and SHAP values are both considered for selecting the top features.

After training, a final machine learning model 122 may be deployed by patient eligibility predictor 124. In example implementations, the final model is in the form of one or more gradient boosted tree models. In one example, the final model uses LightGBM as the framework. It is contemplated, however, that other gradient boosting tree frameworks may be utilized for the final model, such as Spark SQL, CatBoost, or XGBoost.

In aspects, using the final machine learning model 122, patient eligibility predictor 124 determines a plurality of eligibility scores, each indicating a likelihood of whether a target patient is eligible for a bundled payment associated with a particular clinical episode. For example, the eligibility scores may be provided in response to inputting feature values from a target patient’s encounter data and other EHR data. The feature values input into the machine learning model 122 for a target patient are for features that are selected for the machine learning model 122 during feature selection as described above.

Each eligibility score determined by patient eligibility predictor 124 may be a quantitative score, such as a value between 0 and 1. In some aspects, an eligibility score between 0 and 1 may be normalized to a value between 0 and 100. For example, an eligibility score of 0.4 may be normalized to 40 or 40%. In some embodiments, patient eligibility predictor 124 outputs a binary prediction (e.g., yes or no) for whether the target patient qualifies for application of coordinated healthcare resources or a bundled payment for a particular clinical episode. The binary predictions may be determined by applying a threshold to a quantitative score. For example, patient eligibility predictor 124 may output a “no” prediction for any quantitative scores of 0.5 or less and output a “yes” prediction for any quantitative scores that are greater than 0.5. In other implementations, different threshold values may be used. In aspects, the predetermined thresholds for making binary predictions for each of the plurality of bundled payment types may be determined based on historical patient data stored in EHR system 104.

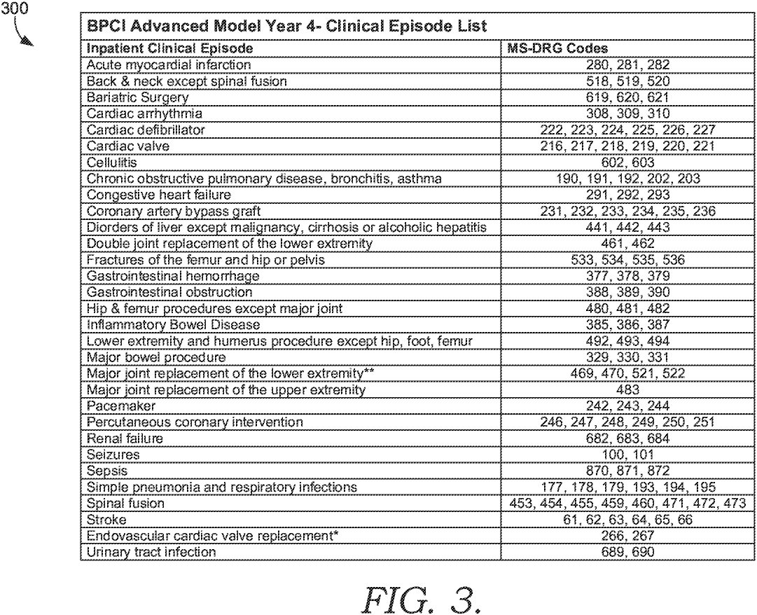

Each eligibility score may be associated with a clinical episode or none of the predetermined clinical episodes. In one example implementation, patient eligibility predictor 124 determines 32 eligibility scores for each target patient: 31 eligibility scores each indicating a likelihood that the target patient qualifies for application of coordinated healthcare resources or a bundled payment under one of the clinical episodes in table 300 in FIG. 3 (e.g., one eligibility score for acute myocardial infarction, one eligibility score for back and neck except spinal fusion, and so on) and one eligibility score indicating a likelihood that the patient does not qualify for bundled payment under one of the 31 clinical episodes.

In some aspects, patient eligibility predictor 124 provides the prediction that the patient qualifies for a particular bundled payment prior to receiving available coding information for determining a DRG for the patient based on the patient encounter. Further, in some aspects, patient eligibility predictor 124 provides the prediction that the patient qualifies for a particular bundled payment prior to the patient being discharged. In this way, patient eligibility predictor 124 provides an early identification of patients eligible for bundled payments. In other words, the patient eligibility may be determined at an early enough stage during care to institute one or more care protocols for providing coordinating care to improve patient outcomes as described further below.

Output provider 126 of the patient eligibility identification system 112 can provide outputs for storage in the database 114 of the patient eligibility identification system 112 and/or for storage in database 106 of the EHR system 104. Further, output provider 126 may provide outputs for display on the user/clinician interface 108. Additionally or alternatively, output provider 126 may output electric signals initiating additional types of actions, such as orders for labs, orders for medications, and/or scheduling as further described below.

In some embodiments, output provider 126 may provide an indication of one or more eligibility scores (either quantitative or qualitative/binary) for a target patient. For example, the outputs may include binary predictions (e.g., yes or no) for whether the target patient qualifies for each clinical episode associated with a bundled payment. Where a binary prediction is output, a qualitative score may also be provided with the binary prediction. In some aspects, one or more thresholds are applied in determining which predictions to output. For example, output provider 126 may indicate potential eligibility only for clinical episodes in which the corresponding eligibility score satisfies a minimum threshold score, such as being greater than or equal to the threshold score. In various implementations, the threshold score may be 0.4, 0.5, 0.6, or 0.8, but it is contemplated that the threshold may be set at another value. In some aspects, the eligibility scores for each class are ranked and only a top number, such as top one class, top three classes, or top five classes, may be output. In either of these ways, output provider 126 may only output indicators of eligibility for certain clinical episodes when there is a relatively greater likelihood of the target patient actually being eligible under those clinical episodes, thereby reducing unnecessary information being provided to a user, such as a clinician.

In some embodiments, output provider 126 outputs only eligibility scores for classes of bundled payments to which a care facility (or healthcare system) subscribes. For example, one facility may not subscribe to bundled payment for congestive heart failure such that determining a patient eligibility for bundled payment for congestive heart failure may not be useful. As such, an eligibility score for this clinical episode may not be output by output provider 126 when being used for a target patient within the facility.

In some embodiments, the indication provided by output provider 126 may be an alert provided to the user/clinician interface 108. This alert may be pushed through to the graphic user interface, may be presented through an electronic communication, such as an email or text message, and/or may be presented as highlighted information when a user opens the target patient’s EHR.

Additionally, in some implementations, output provider 126 may output a recommendation for treating the patient according to a treatment protocol associated with the qualifying bundled payment. As previously described, some care facilities may institute certain protocols when a patient qualifies for a bundled payment to help increase the likelihood of a positive patient outcome and reduce the risk that further services will be needed. Such protocols may include assigning a care manager and/or scheduling the care manager to meet with one or more members of a patient’s care team. Review of the patient’s care plan may be initiated, and the care plan may be revised. Additional discharge instructions may be initiated in some implementations. As such, output provider 126 may output a recommendation for instituting a new or revised care protocol based on the determined patient eligibility for bundled payment for a particular clinical episode.

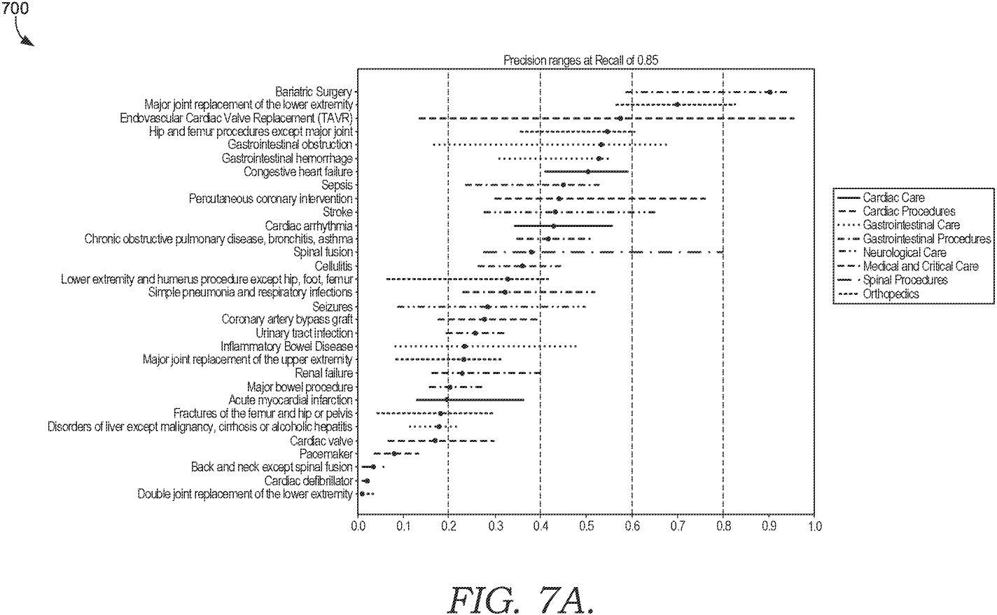

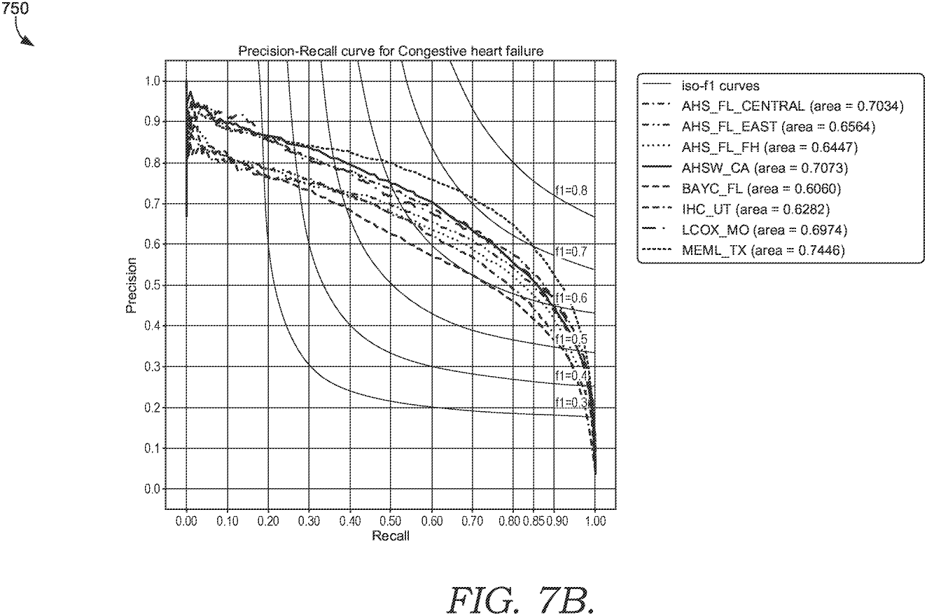

Some aspects of patient eligibility identification system 112 may further include performance analyzer 128 responsible for evaluating the performance of machine learning model 122 utilized for predicting patient eligibility as disclosed herein. Performance analyzer 128 may be implemented during development of machine learning model 122 and/or in deployment of a final machine learning model 122 to determine whether the machine learning model 122 should be updated. The performance indicator determined by performance analyzer 128 may be for an overall model or may be for multiple models, such as for multiple cross-validation models used during model development and/or, in some aspects, models associated with different clinical episodes. In some aspects, performance analyzer 128 determines the performance of machine learning model 122 by determining precision and recall values for one or more models, including the cross-validation models used during model development. FIG. 7A shows an example graphical representation 700 of precision ranges across multiple hospital systems for each clinical episode that were calculated in one example implementation of machine learning model 122. These precision values may be computed by performance analyzer 128, and in some aspects, performance analyzer 128 may output the graphical representation 700 in a graphical user interface, such as on user/clinician interface 108. Performance analyzer 128 may generate precision-recall curves from the precision and recall values. FIG. 7B shows a graphical illustration 750 of precision-recall curves for a model predicting eligibility associated with congestive heart failure for various clients (e.g., healthcare facilities) represented in the data in accordance with one implementation of machine learning model 122. Graphical illustration 750 may also be output by performance analyzer 128 via a graphical user interface, such as on user/clinician interface 108. In embodiments, performance analyzer 128 determines performance indicators of machine learning model 122 for patient subsets associated with gender, race/ethnicity, insurance type, and age. In aspects, python packages (e.g., Pandas, NumPy, SciPy, Scikit-Learn, Matplotlib, SHAP, and Pickle) may be used for determining the performance of the machine learning model 122.

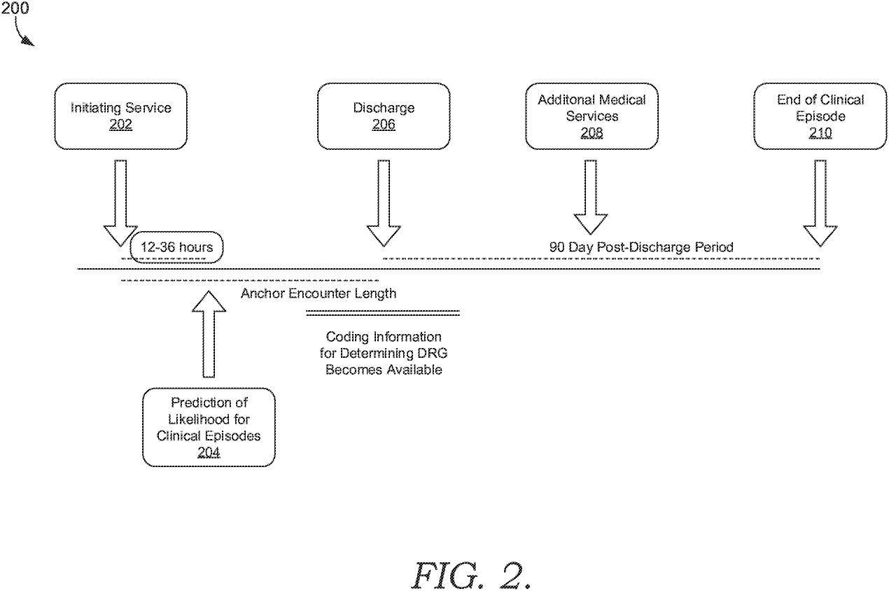

Turning to FIG. 2, FIG. 2 illustrates an example timeline 200 under which aspects of this disclosure may be implemented. In timeline 200, an initiating service 202 for a target patient occurs and may be followed by intermediate actions prior to discharge 206. Initiating service 202 may include, for example, an admission to an acute care facility, an admission to an emergency care facility, or an admission to another healthcare facility. In some aspects, in response to the initiating service 202, a patient eligibility prediction 204 (e.g., a prediction of a likelihood for clinical episodes associated with bundled payment) may be provided, via a graphical user interface, for the target patient. The patient eligibility prediction may indicate (via the graphical user interface of a clinician user interface such as user/clinician interface 108, for example) that the target patient qualifies for one or more of a plurality of bundled payments each associated with a clinical episode. In a non-limiting example, the patient eligibility prediction 204 may be provided within 12-36 hours after initiating service 202.

In aspects, an anchor encounter length comprises the time from initiating service 202 to the time of discharge 206. As depicted by the timeline 200, a patient eligibility predictor may programatically determine and provide the patient eligibility prediction 204 for the target patient after initiating service 202 and prior to discharge 206. In various aspects, the patient eligibility prediction 204 may include an indication (e.g., via the graphical user interface) to initiate a care protocol associated with a bundled payment for which the patient qualifies. Furthermore, as depicted by timeline 200, the patient eligibility prediction 204 may be provided prior to receiving available coding information for determining a DRG for the patient.

Additionally, timeline 200 illustrates that the patient eligibility prediction 204 may be provided prior to a post-discharge period. As depicted by example timeline 200, the post-discharge period may be a 90-day period that begins upon or after discharge 206. Additionally, the post-discharge period may start the day that the anchor encounter length ends. Further, during the post-discharge period, additional medical services 208 may be provided to the target patient. One example of an additional medical service 208 may include an end-stage renal disease service performed during the post-discharge period (e.g., self-care dialysis). In some aspects, the additional medical service 208 provided may be an included service within the bundled payment predicted by the patient eligibility prediction 204. In some aspects, the end of the 90-day post-discharge period marks the end of the clinical episode 210.

Turning to FIG. 3, example table 300 illustrates an example listing of types of clinical episodes for which bundled payments may be available. The types of clinical episodes listed in table 300 may represent classes for which a patient eligibility predictor, such as patient eligibility predictor 124 of FIG. 1, determines an eligibility score. Table 300 further shows an example of how these clinical episodes types may be defined by MS-DRGs. For example, a gastrointestinal hemorrhage inpatient clinical episode may be associated with MS-DRG codes 377, 378, and 379. The left column of table 300 provides a list of example clinical episodes and the right column of table 300 provides one or more MS-DRG codes for each of the corresponding clinical episodes. In aspects of training a machine learning model to predict target patient’s qualification for a bundled payment, the MS-DRG codes may be used to label historical patient encounters stored in an EHR system with a clinical episode. In some aspects, the labels are created based on information from the MS-DRG codes, a table of claims stored within an EHR system, or one or more combinations thereof. In some embodiments, the machine learning model can be trained using claims generated by a billing module that is part of the EHR or interfacing with the EHR. In some embodiments, the machine learning model can be trained using claims data obtained from payors and associated with data stored in the EHR. In some embodiments, the EHR data is retrieved via HealtheIntent.

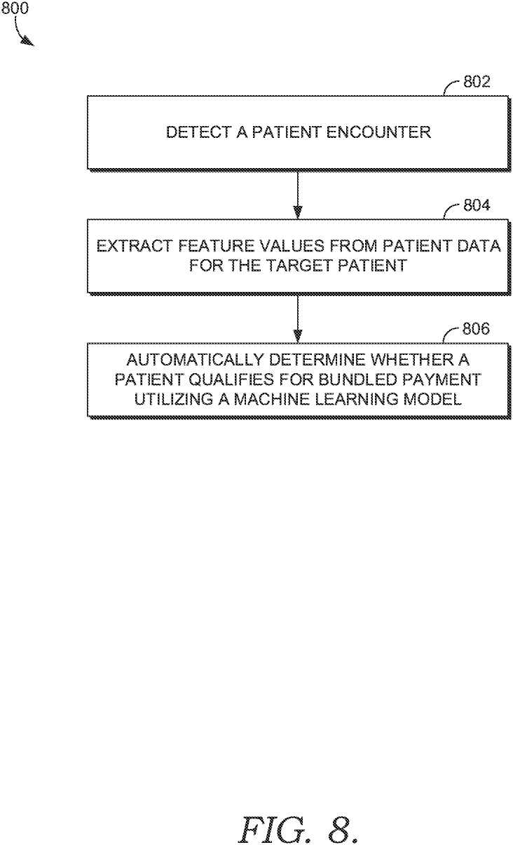

Turning to FIG. 8, flow diagram depicts an example method 800 for a patient eligibility predictor. Method 800 may be implemented by one or more components of patient eligibility identification system 112 of FIG. 1. Beginning with step 802, the method includes detecting a patient encounter associated with a target patient. The target patient is a patient for which eligibility is sought to be determined. In aspects, the patient encounter may include an inpatient encounter or an outpatient encounter. In some aspects, detection of the patient encounter is based on receiving an indication of the patient encounter (e.g., by an entry into an EHR system via a user or a sensor, such as a medical device receiving signals that are subsequently transmitted to the EHR system). In some embodiments, detection of the patient encounter is based scanning EHR data to identify the target patient using associated patient data (e.g., that was reviewed by a medical practitioner, such as a physician for example).

Turning to step 804, the method includes extracting feature values for a patient from an EHR system. Step 804 may be performed by an embodiment of extractor 120 of FIG. 1. In aspects, the feature values are associated with an EHR of the patient stored in the EHR system. The EHR system may also store EHRs of a plurality of other patients. Prior to storing the EHRs in the EHR system, the EHR system may have received the EHRs from a plurality of healthcare facilities. In aspects, the extracted feature values are for features selected during training of a machine learning model. These features may be administrative data (e.g., information about past and current encounters), demographics, conditions, medications, lab results, and/or vitals features.

Turning to step 806, the method provides for inputting the extracted feature values into a machine learning model to determine whether the target patient qualifies for bundled payment. Step 806 may be performed by an embodiment of patient eligibility predictor 124 of FIG. 1. As bundled payment is associated with types of clinical episodes, step 806 may comprise determining the likelihood the encounter of the target patient is a particular clinical episode associated with bundled payment. In some embodiments, the machine learning model, which may be an embodiment of machine learning model 122 of FIG. 1, is a multiclass classification model that determines a plurality of eligibility scores, each indicating the likelihood that the target patient qualifies for bundled payment for a particular clinical episode. For example, the determination may comprise determining a first likelihood that the target patient qualifies for the bundled payment for a first clinical episode (which could be a procedure-based clinical episode) and a second likelihood that the target patient qualifies for the bundled payment for a second clinical episode (which could be a diagnosis-based episode clinical episode).

In some aspects, the method 800 may further include initiating one or more actions based on a determination that the patient qualifies for bundled payment, which may be done by output provider 126 of FIG. 1. In one embodiment, the action may include causing for display, on a graphical user interface, an indication that the target patient qualifies for the bundled payment. This indication may be generally for one or more particular clinical episodes. For example, in some aspects, the method 800 may cause the graphical user interface to display an indication that the target patient qualifies for bundled payment for a first clinical episode and a second episode for a single patient encounter. The indication may include a ranking of the different clinical episodes based on the associated eligibility scores (e.g., likelihoods) for the target patient. In some aspects, only the rankings for the bundled payments for which the target patient qualifies are displayed. For example, in one embodiment, the machine learning model may determine 32 eligibility scores for 31 clinical episodes and one “other” or “none”, but rankings for bundled payments may only be displayed for clinical episodes having associated eligibility scores that satisfy a threshold.

Further, in some aspects, the application of coordinated healthcare resources initiated based on programatically determining that the target patient qualifies for particular healthcare resources may include assigning a care manager to the target patient/or scheduling time with a care manager. The care protocol may include, additionally or alternatively, instituting specialized discharged instructions and/or requiring additional testing or examination prior to discharge. In some aspects, the care protocol may include scheduling a follow-up appointment for the target patient. In some aspects, the application of coordinated healthcare resources may include allocating a particular treatment or treatment plan to the target patient or including the target patient in a group of patients who are to receive the particular treatment or treatment plan.

Some aspects of the disclosure further include operations for training the machine learning model utilized in method 800. The machine learning model may be trained utilizing historical patient data for a reference population of patients. Training data selection criteria may be applied to identify an appropriate training cohort. Each encounter within the historical patient data may be labeled with a clinical episode to be used during training. This step may include mapping historical patient data of patients to a standardized code associated with a clinical episode. In some embodiments, the standardized code may comprise MS-DRG codes.

Further, some aspects of training include applying determining feature metrics using cross-validation models to determine feature importance and select features for implementation in a final machine learning model. In some aspects, feature selection may include separating features of the historical patient data into binary and non-binary (e.g., continuous) feature sets. One or more gradient boosted tree models may be used for the models during feature selection. Different sets of features may be selected for each class (e.g., clinical episode) for which the machine learning model makes a prediction.

Turning to FIG. 9, there is shown one example embodiment of computing system 900 representative of a system architecture that is suitable for computer systems (such as computer system 110 depicted in FIG. 1, for example). Computing system 900 includes a bus 902 that directly or indirectly couples the following devices: memory 904, one or more processors 906, one or more presentation components 908, input/output (I/O) ports 910, input/output components 912, radio 914, and an illustrative power supply 916. Bus 902 represents what may be one or more busses (such as an address bus, data bus, or combination thereof). Although the various blocks of FIG. 9 are shown with lines for the sake of clarity, in reality, delineating various components is not so clear, and metaphorically, the lines would more accurately be grey and fuzzy. For example, one may consider a presentation component, such as a display device, to be an I/O component. Also, processors have memory. As such, the diagram of FIG. 9 is merely illustrative of an exemplary computing system that can be used in connection with one or more embodiments of the present invention. Distinction is not made between such categories as “workstation,” “server,” “laptop,” “hand-held device,” etc., as all are contemplated within the scope of FIG. 9 and reference to “computing system.”

Computing system 900 typically includes a variety of computer-readable media. Computer-readable media can be any available media that can be accessed by computing system 900 and includes both volatile and nonvolatile media, and removable and non-removable media. By way of example, and not limitation, computer-readable media may comprise computer storage media and communication media. Computer storage media includes both volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computing system 900. Computer storage media does not comprise signals per se. Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

Memory 904 includes computer storage media in the form of volatile and/or nonvolatile memory. The memory may be removable, non-removable, or a combination thereof. Exemplary hardware devices include solid-state memory, hard drives, optical-disc drives, etc. Computing system 900 includes one or more processors that read data from various entities such as memory 904 or I/O components 912. Presentation component(s) 908 present data indications to a user or other device. Exemplary presentation components include a display device, speaker, printing component, vibrating component, etc.

In some embodiments, computing system 900 comprises radio(s) 914 that facilitates communication with a wireless-telecommunications network. Illustrative wireless telecommunications technologies include CDMA, GPRS, TDMA, GSM, and the like. Radio 914 may additionally or alternatively facilitate other types of wireless communications including Wi-Fi, WiMAX, LTE, or other VoIP communications. As can be appreciated, in various embodiments, radio 914 can be configured to support multiple technologies and/or multiple radios can be utilized to support multiple technologies.

I/O ports 910 allow computing system 900 to be logically coupled to other devices, including I/O components 912, some of which may be built in. Illustrative components include a microphone, joystick, game pad, satellite dish, scanner, printer, wireless device, etc. The I/O components 912 may provide a natural user interface (NUI) that processes air gestures, voice, or other physiological inputs generated by a user. In some instances, inputs may be transmitted to an appropriate network element for further processing. An NUI may implement any combination of speech recognition, stylus recognition, facial recognition, biometric recognition, gesture recognition both on screen and adjacent to the screen, air gestures, head and eye tracking, and touch recognition (as described in more detail below) associated with a display of the computing system 900. The computing system 900 may be equipped with depth cameras, such as stereoscopic camera systems, infrared camera systems, RGB camera systems, touchscreen technology, and combinations of these, for gesture detection and recognition. Additionally, the computing system 900 may be equipped with accelerometers or gyroscopes that enable detection of motion.

The architecture depicted in FIG. 9 is provided as one example of any number of suitable computer architectures, such as computing architectures that support local, distributed, or cloud-based software platforms, and are suitable for supporting computer system 110.

Many different arrangements of the various components depicted, as well as components not shown, are possible without departing from the spirit and scope of the present invention. It will be understood that certain features and sub-combinations are of utility and may be employed without reference to other features and sub-combinations and are contemplated within the scope of the claims. Not all steps listed in the various figures need be carried out in the specific order described. Accordingly, the scope of the invention is intended to be limited only by the following claims.

What is claimed is:

1. Non-transitory computer storage media having computer-executable instructions embodied thereon that, when executed on a computerized decision support system, perform operations comprising:
detecting a patient encounter associated with a target patient;
extracting feature values from patient data in an electronic health record of the target patient; and
programatically determining, utilizing a machine learning model, that the target patient qualifies for application of coordinated healthcare resources based on feature values extracted from the patient data.

2. The media of claim 1, wherein the machine learning model comprises a multiclass classification model, and wherein programatically determining that the target patient qualifies for the application of coordinated healthcare resources comprises:
determining a plurality of eligibility scores, each of the plurality of eligibility scores being associated with a clinical episode and indicating a likelihood that the target patient qualifies for the application of coordinated healthcare resources for a corresponding clinical episode.

3. The media of claim 2, further comprising:
determining that a first set of the plurality of eligibility scores indicate a higher likelihood that the target patient qualifies for the application of coordinated healthcare resources than a second set of the plurality of eligibility scores; and
programatically determining that the target patient qualifies for the application of coordinated healthcare resources for clinical episodes associated with the first set of the plurality of eligibility scores.

4. The media of claim 1, wherein the application of coordinated healthcare resources comprises allocating a particular treatment or treatment plan to the target patient.

5. The media of claim 4, wherein the operations further comprise causing to display, on a graphical user interface, an indication that the target patient qualifies for the application of coordinated healthcare resources.

6. The media of claim 1, the operations further comprising initiating a protocol associated with a bundled payment for the target patient based on programatically determining that the target patient qualifies for application of coordinated healthcare resources.

7. The media of claim 1, wherein programatically determining that the target patient qualifies for application of coordinated healthcare resources comprises determining a likelihood that the target patient qualifies for application of coordinated healthcare resources based on at least one of a plurality of clinical episodes, and wherein the operations further comprise causing to display, on a graphical user interface, an indication of a clinical episode for which the target patient qualifies.

8. The media of claim 7, wherein programatically determining that the target patient qualifies for application of coordinated healthcare resources comprises determining that the target patient qualifies for application of coordinated healthcare resources based on at least two of the plurality of clinical episodes, and wherein the operations further comprise causing to display, on the graphical user interface, a ranking of each clinical episode for which the target patient qualifies.

9. The media of claim 1, wherein the machine learning model comprises a binary classification model.

10. The media of claim 1, wherein the feature values comprise values for administrative data, demographics, conditions, medications, lab results, and vitals features.

11. The media of claim 1, wherein the operations further comprise training the machine learning model by:
receiving historical patient data of a reference population of patients, the historical patient data associated with clinical episodes;
extracting feature values associated with patient encounters from the historical patient data;
labeling the patient encounters within the historical patient data with a clinical episode; and
training the machine learning model based on the extracted feature values and the labeling of the patient encounters.

12. The media of claim 11, wherein labeling the patient encounters in the historical patient data includes mapping the historical patient data to a standardized code.

13. A computerized patient eligibility predictor comprising:
one or more processors;
memory storing computer-usable instructions that, when executed by the one or more processors, perform operations comprising:
receiving an indication of a patient encounter associated with a target patient;
extracting feature values from an electronic health record associated with the target patient; and
programatically determining, utilizing a machine learning model, at least one eligibility score indicating whether the target patient qualifies for application of coordinated healthcare resources based on the extracted feature values.

14. The computerized patient eligibility predictor of claim 13, wherein determining at least one eligibility score indicating whether the target patient qualifies for application of coordinated healthcare resources comprises:
determining, utilizing the machine learning model, an eligibility score for each of a plurality of clinical episodes associated with application of coordinated healthcare resources;
ranking each of the plurality of clinical episodes based on the eligibility score; and
providing at least one of the plurality of clinical episodes to a graphical user interface for display, the at least one of the plurality of clinical episodes having a highest ranking.

15. The computerized patient eligibility predictor of claim 14, wherein the operations further comprise:
determining that the target patient qualifies for application of coordinated healthcare resources by comparing the eligibility scores for each of the plurality of clinical episodes to a threshold score to determine that the target patient qualifies for application of coordinated healthcare resources based on at least one clinical episode; and
displaying, on a graphical user interface, an indication that the target patient qualifies for application of coordinated healthcare resources based on the at least one clinical episode.

16. The computerized patient eligibility predictor of claim 14, wherein the machine learning model comprises a multiclass classification model, and wherein the operations further comprise:
ranking each of the plurality of clinical episodes based on the eligibility score associated with each clinical episode; and
displaying, on a graphical user interface, the ranking for at least a subset of the plurality of clinical episodes.

17. A computerized method for programatically determining whether a target patient qualifies for application of coordinated healthcare resources, the method comprising:
detecting a patient encounter associated with the target patient;
extracting feature values from patient data in an electronic health record for the target patient; and
programatically determining, utilizing a machine learning model, that the target patient qualifies for application of coordinated healthcare resources based on the extracted feature values.

18. The computerized method of claim 17, wherein programatically determining that the target patient qualifies for the application of coordinated healthcare resources occurs prior to discharge.

19. The computerized method of claim 17, wherein the patient encounter is detected by scanning the electronic health record data to identify the target patient and associated patient data that was reviewed by a medical practitioner.

20. The computerized method of claim 17, wherein programatically determining that the target patient qualifies for application of coordinated healthcare resources comprises determining, utilizing the machine learning model, a plurality of eligibility scores for a plurality of clinical episodes associated with application of coordinated healthcare resources, each eligibility score indicating a likelihood that the target patient qualifies for application of coordinated healthcare resources based on a particular clinical episode from the plurality of clinical episodes.
